(19) United States
(12) Patent Application Publication
(10) Pub. No.: US 2026/0055356 A1
(43) Pub. Date: Feb. 26, 2026

(54) CELL CULTURE SYSTEM WITH ALTERED CELLULAR MICROGRAVITY AND SHEAR STRESS

(71) Applicant: THE REGENTS OF THE UNIVERSITY OF CALIFORNIA, Oakland, CA (US)

(72) Inventors: Derfogail Delcassian, Oakland, CA (US); Emily Adler, Oakland, CA (US); Lordean Gustinvil, Berkeley, CA (US); Saheer Kamal Ahmed, San Jose, CA (US)

(21) Appl. No.: 19/362,859

(22) Filed: Oct. 20, 2025

Related U.S. Application Data
(63) Continuation of application No. PCT/US2024/026173, filed on Apr. 25, 2024.
(60) Provisional application No. 63/498,105, filed on Apr. 25, 2023.

(51) Int. Cl. C12M 1/42; C12M 1/12; C12M 1/32; C12M 3/00

(52) U.S. Cl. CPC C12M 35/04; C12M 23/06; C12M 23/12; C12M 23/44; C12M 23/48

(57) ABSTRACT

A rotary cell culture system (RCCS) includes at least one concentric ring attached to a frame along an axis of rotation of the at least one concentric ring, and a DC motor coupled to the at least one concentric ring via one or more gears and configured to rotate the at least one concentric ring along the axis. The RCCS may include at least two concentric rings, wherein each ring of the at least two concentric rings is attached to an adjacent ring such that the axis of rotation of the at least one concentric ring is orthogonal to an axis of rotation of any other of the concentric rings. An inner ring of the RCCS is configured to hold one or more sample holding members or structures such as cell culture tubes or a micro well plate structure.

### CROSS-REFERENCE TO RELATED APPLICATIONS

This application is a continuation of International Application Serial No. PCT/US2024/026173, filed Apr. 25, 2024, which claims priority to U.S. Provisional Application No. 63/498,105, filed on Apr. 25, 2023, the disclosures of which are each hereby incorporated by reference in its entirety.

### BACKGROUND

The present disclosure provides systems and methods for microgravity emulation.

Long term space exploration and commercialized space travel are becoming increasingly popular ideas as advancements in space technology make the journey safer for all, regardless of their occupation and training. However, recent observations show that over half of returning astronauts experience infections, colds, or the reactivation of dormant viruses within a week of returning to Earth (Rooney B V. Fro Microbiol. 2019; 10:16.).

Additionally, microgravity may alter cellular processes including growth and cell organization. Accessing and manipulating cells in these unique cell states is currently challenging. Developing a system which facilitates study of cell behaviour and cellular processes, or facilitates cellular manipulation in conditions of altered microgravity and/or fluidic shear forces would be useful.

Accordingly it would be desirable to study the effects of gravity on biological processes, including for example regulatory T cell (Treg) activation and seed germination. It would also be desirable to study the impact of delivering nucleic acids to cells (or cell aggregation of cells) in these unique environments.

### SUMMARY

The present embodiments provide a multi-axis rotary cell culture system (RCCS) and uses thereof. In certain embodiments, the RCCS may be 3D-printed and low-cost. The RCCS may be used to study Regulatory T cell (Treg) activation, and other biological processes and behaviors, within a simulated microgravity (μG) environment. The RCCS may also be used to study the impact of delivering nucleic acids to cells (or cell aggregation of cells) in these unique environments.

The RCCS advantageously enables the study of cellular behaviour in altered gravity (microgravity) and/or with varied fluid shear stress forces experienced by cells, and/or in a variety of cell culture containers, and/or whole organisms.

According to an embodiment, a multi-axis rotary cell culture system (RCCS), comprising up to three concentric rings is provided, wherein an inner ring is configured to hold one or more cell culture tubes, and wherein each ring rotates around an axis orthogonal to the axes of the other rings.

According to certain aspects, the inner ring is removable and interchangeable.

According to certain aspects, the RCCS further includes a DC motor coupled to an outer ring via one or more gears.

According to certain aspects, the RCCS further includes a speed controller configured to control operation of the DC motor and rotation of the three rings.

According to certain aspects, the three concentric rings are attached to each other at opposing axes connection points.

According to certain aspects, an outer ring of the three concentric rings is attached to a frame along an axis of rotation of the outer ring.

According to certain aspects, the RCCS further includes a DC motor coupled to the outer ring via one or more gears and configured to rotate the outer along an axis of the outer ring.

According to an embodiment, a rotary cell culture system (RCCS) is provided that includes at least one concentric ring attached to a frame along an axis of rotation of the at least one concentric ring, and a DC motor coupled to the at least one concentric ring via one or more gears and configured to rotate the at least one concentric ring along the axis.

According to certain aspects, the at least one concentric ring is configured to hold one or more cell culture tubes.

According to certain aspects, the RCCS includes at least two concentric rings, wherein each ring of the at least two concentric rings is attached to an adjacent ring such that the axis of rotation of the at least one concentric ring is orthogonal to an axis of rotation of any other of the concentric rings.

According to certain aspects, an inner ring of the at least two concentric rings is configured to hold one or more cell culture tubes.

According to certain aspects, the inner ring is removable and interchangeable.

According to certain aspects, the RCCS includes three concentric rings.

According to an embodiment, a method of processing biological material in a microgravity environment using a rotary cell culture system (RCCS) is provided.

According to an embodiment, a method of creating a microgravity environment using a rotary ring system having at least on ring configured to rotate about a first axis is provided. The method typically includes placing a sample, within, e.g., proximal to a center point or a periphery, of the at least one ring; and rotating the at least one ring about the first axis.

In certain aspects, the method uses any of the multi-axis ring system embodiments as described herein.

According to an embodiment, a method of processing biological material in a microgravity environment using a rotary cell culture system (RCCS) having at least on ring configured to rotate about a first axis is provided. The method typically includes placing a biological sample within, e.g., proximal to a center point, of the at least one ring; and rotating the at least one ring about the first axis.

In certain aspects, the RCCS further includes a second ring configured to rotate about a second axis orthogonal to the first axis, wherein a diameter of the second ring is greater than a diameter of the first ring, and wherein the first ring is connected to the second ring at opposing axes connection points, wherein rotating the at least one ring includes rotating the second ring.

In certain aspects, the RCCS further includes a second ring and a third ring configured to rotate about second and third axes, respectively, wherein the second axis is orthogonal to both the first axis and the second axis, wherein a diameter of the third ring is greater than a diameter of the first ring and a diameter of the second ring, and wherein the diameter of the second ring is greater than the diameter of the first ring, and wherein the first ring is connected to the second ring at opposing axes connection points, wherein the second ring is connected to the third ring at opposing axes connection points, wherein the third ring is connected to a frame at opposing axes connection points, and wherein rotating the at least one ring includes rotating the third ring.

In certain aspects, the biological sample includes one of a seed, plant material, or a seed and a nutrient rich substrate.

In certain aspects, the RCCS further includes a sample holding structure located proximal to a center of the inner ring and held by a structure coupled with or integrated into the inner ring.

In certain aspects, an inner ring of the at least two concentric rings is configured to hold a sample holding structure proximal to a center of the inner ring.

In certain aspects, the sample holding structure includes a micro well plate structure.

In certain aspects, the RCCS further includes a stand with adjustable connector elements configured to change an angle of the at least one concentric to enable a range of different gravitational environments.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE SEVERAL VIEWS OF THE DRAWING(S)

FIG. 1A illustrates a CAD schematic of an RCCS embodiment shown in two different orientations, according to an embodiment.

FIG. 1B shows a single ring RCCS, according to an embodiment.

FIG. 1C shows a dual ring RCCS, according to an embodiment.

FIG. 1D shows a three ring RCCS, according to an embodiment.

FIG. 1E is a schematic of 3D Pythagoras theorem incorporating x, y and z dimensions used to calculate resultant gravity.

FIG. 1F shows a wireless, Bluetooth enabled accelerometer attached to an origin point.

FIG. 1G, FIG. 1H and FIG. 1I show graphs displaying measured g in the x, y and z direction to demonstrate nullification of gravity vectors.

FIG. 1J shows an embodiment with three concentric rings.

FIG. 2, panel A is a schematic of a 2-ring device including a 3D printed polymer transmission belt according to an embodiment.

FIG. 2, panel B shows images of a 3D printed and assembled system with a dual tube holder as the central ring.

FIG. 2, panel C shows vector positional data in the x, y, and z direction.

FIG. 2, panel D shows the resultant average g experienced by the accelerometer over 2 hours.

FIG. 2, panels E and F show accelerometer data measured in 2-ring geared systems at various RPM speeds, with an accelerometer attached at 0, 0.5, 1.5, 3, or 6 cm from the origin along an axis at 90°to the attachment point (yellow line in FIG. 2, panel E).

FIG. 2, panel F shows resultant acceleration from three independent experiments run at 10 RPM for 15 minutes.

FIG. 2, panel G shows representative plots of nullification of gravity.

FIG. 2, panel H shows resultant acceleration from three independent experiments run at 1, 3, 10, 25 or 45 RPM.

FIG. 2, panel I shows graphical representations of acceleration in the x, y and z directions at 45 RPM. This data indicates that these devices can advantageously maintain microgravity around 0.1 g up to 6 cm from the origin at lower speeds.

FIG. 3, Panels A-G illustrate a comparison of an embodiment to commercially available technologies.

FIG., panels A-C show interchangeable inner ring design embodiments for versatile dynamic cell culture systems.

FIG. 4, panels D-F show vector positional data measured using an accelerometer attached at the indicated red dot on the devices of FIGS. 4A, 4B, and 4C, respectively.

FIG. 5, panels A-H show successful aggregation and transfection of beta cell aggregates using an embodiment (“RPM”), and a comparison to current technologies (Aggrewell aggregation and Orbital Shaker transfection).

FIG. 6, panel A is a schematic of cellular activation studies performed in a microgravity device embodiment, under static gravity conditions and using oxygen control conditions.

FIG. 6, panel B shows a comparison of CD69 expression in T cells stimulated in microgravity, under static gravity conditions and using oxygen control conditions.

FIG. 7 shows images of different configurations for a microgravity device using different stand configurations according to embodiments.

FIG. 8 shows a design for an inner ring with a sample holding structure, e.g., plate or block-shaped holder, integrated in the inner ring, according to an embodiment.

FIG. 9 shows design schematics of various possible example structural modifications to aid stability.

FIG. 10 illustrates aspects of a biological proof of concept study that shows seed treatment in microgravity enhances crop biomass and root growth across species.

### DETAILED DESCRIPTION

According to embodiments, novel, low-cost, multi-axis rotary cell culture systems (RCCSs) are provided. In certain embodiments, the devices may be entirely or partially 3-D printed.

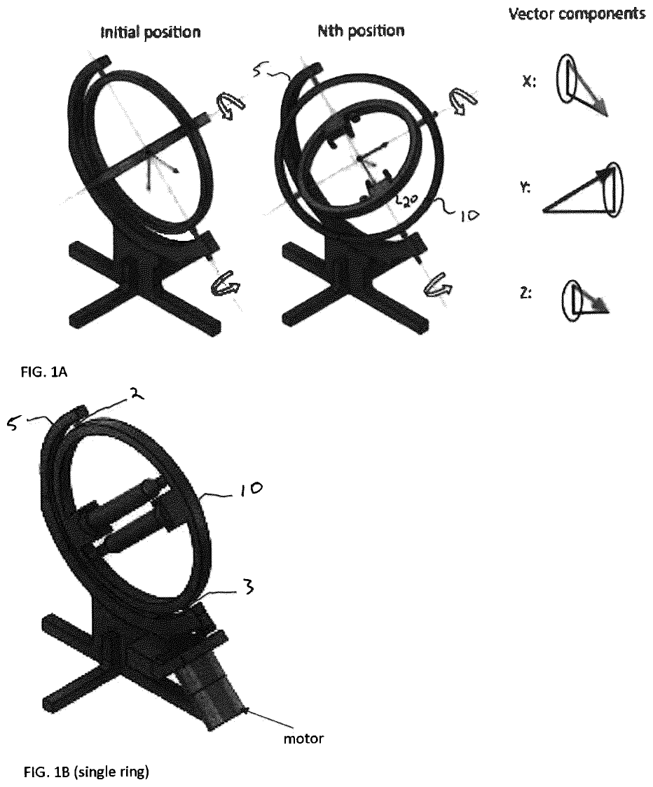

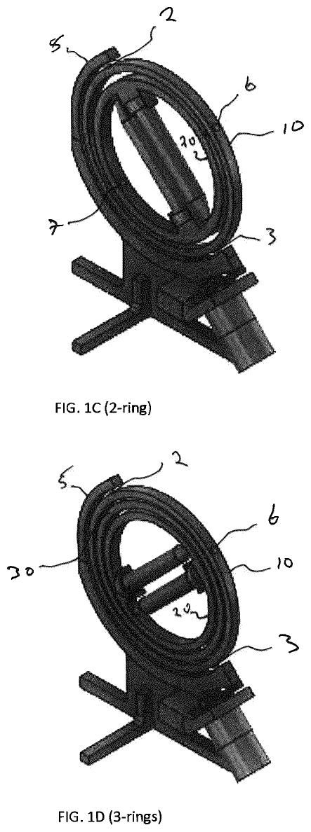

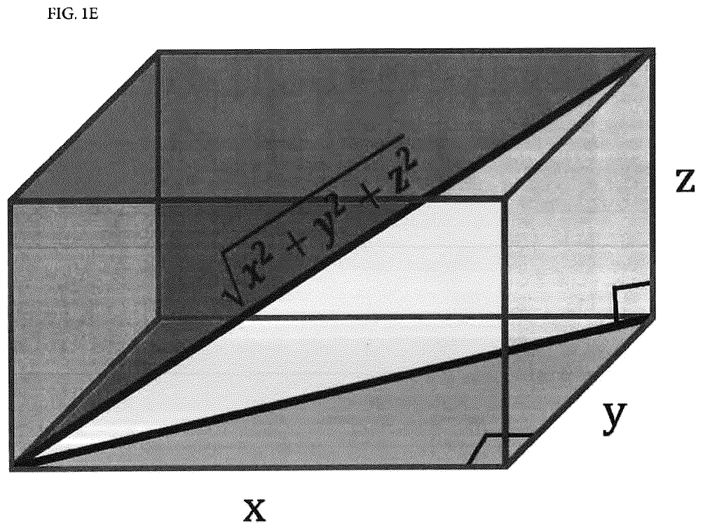

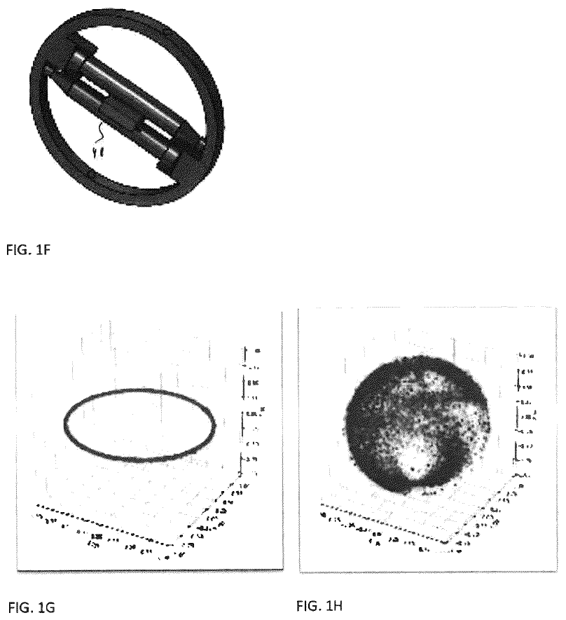

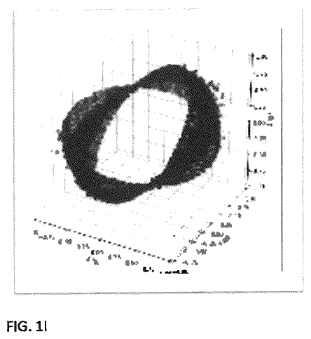

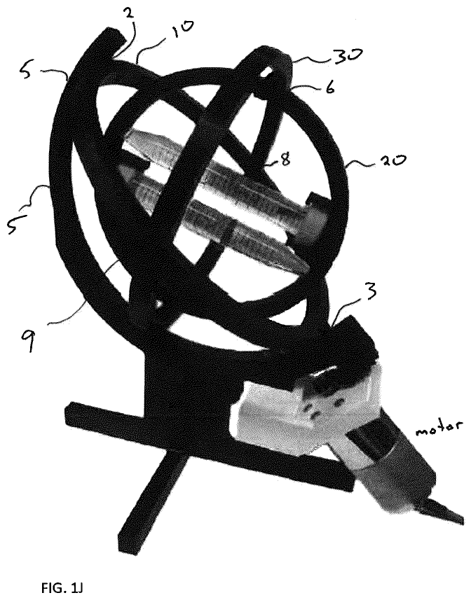

FIG. 1A illustrates a CAD schematic of a device embodiment shown in two different orientations; serial rings are connected, shown here at 90°to each other, allowing random rotation around attachment pivot points, where x, y and z vector components are indicated at various device rotation positions.

In embodiments, the RCCS design may include one two or three rings attached at opposing axes to allow for 360°rotation about the centre of mass as shown in FIGS. 1B, 1C and 1D. More rings may be used. For example, FIG. 1B shows a single ring embodiment, with the single ring 10 coupled with a motor (e.g., DC motor) that is configured to drive the ring 10 (e.g., via a belt or other mechanism) to rotate about its axis. The axis is defined based on the connection points 2 and 3 connecting ring 10 to frame 5. Connection points to the frame may include a pin or other mechanism that enables a ring to rotate about its axis as defined by the connection points, i.e., such that the ring 10 is rotatably attached to the frame 5. In an embodiment the axes of each of outer ring 10 and inner ring 20 are orthogonal to each other. Similarly, FIG. 1C shows a dual ring embodiment, with the outer ring 10 coupled with a motor (e.g., DC motor) that is configured to drive the outer ring 10 (e.g., via a belt or other mechanism) to rotate about its axis. The inner ring 20 rotates about its axis, which is defined based on the connection points 6 and 7 to outer ring 10. Connection points between rings may include a pin or other mechanism that enables a ring to rotate about its axis as defined by the connection points, i.e., such that attached rings are rotatably attached to each other. Similarly, FIG. 1D shows a three ring embodiment, with the outer ring 10 coupled with a motor (e.g., DC motor) that is configured to drive the outer ring 10 (e.g., via a belt or other mechanism) to rotate about its axis. The inner ring 20 rotates about its axis, which is defined based on the connection points 6 and 7 to intermediary ring 30. The intermediary ring 30 rotates about its axis, which is defined based on the connection points 8 and 9 (see FIG. 1J) to outer ring 10. In an embodiment the axes of each of outer ring 10, inner ring 20 and intermediary ring 30 are orthogonal to each other.

In certain embodiments, a small gear is coupled to a DC motor that drives a larger gear attached to the outer ring 10 to rotate about the θ axis. In this example, a controller (e.g., Arduino controller) including a processor and associated memory and communication interface(s) is attached to the motor connected to the outermost ring 10. The central or inner ring in each of these devices may be designed to support desired components such as an accelerometer or one or more tubes. For example, FIGS. 1B, 1C and 1D each shows images of a device configured to hold two containers, e.g., 15 ml Falcon tubes. The speed at which the outer ring 10 rotates can be adjusted through the speed controller allowing for the nullification of gravitational forces over time, e.g., by centrifugal force, and Coriolis force acting upon the cells within the inner ring 20. The speed controller includes a processor and associated memory storing instructions to control the DC motor, automatically, or in response to received control signals. It is noted that in single ring embodiments, the outer ring 10 is the same as the inner ring, and hence “10” and “20” may be used interchangeable in single ring embodiments to denote the “inner ring”.

FIG. 1E is a schematic of 3D Pythagoras theorem incorporating x, y and z dimensions used to calculate resultant gravity. FIG. 1F shows a schematic of a wireless, Bluetooth enabled accelerometer attached to the origin point of the central or inner ring 20 of 1,2 and 3-ring devices. In a study, devices were allowed to randomly rotate with motor input set to 10 RPM for 15 minutes. Acceleration in each axis of the vector components was measured and the resultant mean gravity vector was calculated. FIGS. 1G, 1H and 1I show graphs displaying measured g in the x, y and z direction to demonstrate nullification of gravity vectors. Each graph includes all data points collected during a 15 minute run from one representative experiment at 10 RPM for each of the 1, 2 and 3-ring systems.

FIG. 1J shows an embodiment with three concentric rings. For one-, two-, or three-ring embodiments, an outer ring 10 is coupled or attached via connection points to a frame along an axis of rotation of the outer ring 10, and a DC motor is coupled to the outer ring 10 via one or more gears and is configured to rotate the outer ring 10 along the axis of rotation of the outer ring. As shown, each of the three concentric rings is attached to an adjacent ring at opposing axes ends, and each ring is able to rotate around an axis orthogonal to the axes of an adjacent ring. Also, the inner ring 20 is configured to hold one or more cell culture tubes. For single ring embodiments, the single ring is both the inner ring and the outer ring. The axis of rotation of the outer ring is always fixed relative to the frame. For two-ring embodiments, the two rings rotate along orthogonal axes. For three ring embodiments, the axis of rotation of the inner ring 20 may at times be parallel to the axis of rotation of the outer ring 10, or may have a component parallel to the axis of rotation of the outer ring.

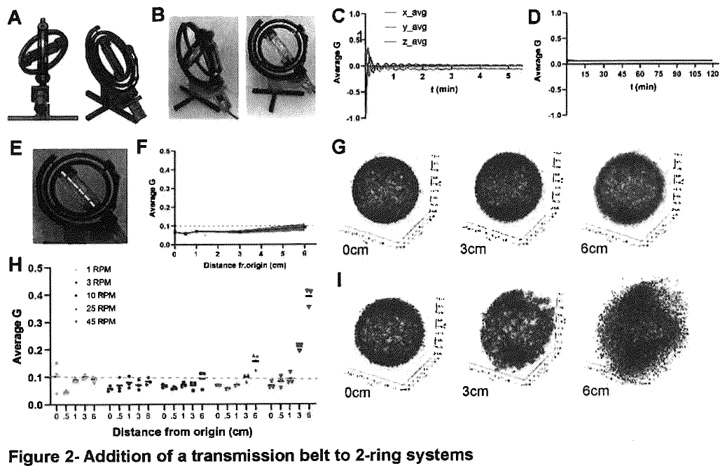

FIG. 2, panel A is a schematic of a 2-ring device including a 3D printed polymer transmission belt according to an embodiment. As shown, serial rings are connected, with a transmission belt connecting the outer ring to the inner ring. Rings shown here are connected at 90°to each other using a 1:1 gearing ratio from the motor to the central ring. The transmission belt in this example is 3D printed from a thermo-setting filament, which facilitates heat-recovery of the transmission belt to return it to its optimal designed shape should wear or stretch occur. FIG. 2, panel B shows images of a 3D printed and assembled system with a dual 15 ml Falcon tube holder as the central ring. FIG. 2, panel C shows vector positional data in the x, y, and z direction measured over 5 minutes with an accelerometer attached to the center origin of the device run at 10 RPM for up to 2 hours; FIG. 2, panel D shows the resultant average g experienced by the accelerometer over 2 hours. FIG. 2, panel F shows accelerometer data measured in 2-ring geared systems at various RPM speeds, with an accelerometer attached at 0, 0.5, 1.5, 3, or 6 cm from the origin along an axis at 90°to the attachment point (yellow line in FIG. 2, panel E). FIG. 2, panel F shows resultant acceleration from three independent experiments run at 10 RPM for 15 minutes, and FIG. 2, panel G shows representative plots of nullification of gravity. FIG. 2, panel H shows resultant acceleration from three independent experiments run at 1, 3, 10, 25 or 45 RPM and FIG. 2, panel I shows graphical representations of acceleration in the x, y and z directions at 45 RPM. This data indicates that this embodiment of the device can advantageously maintain microgravity around 0.1 g up to 6 cm from the origin at lower speeds. (additional data from other embodiments as discussed below maintains gravity <0.05 G (˜5% Earth G).

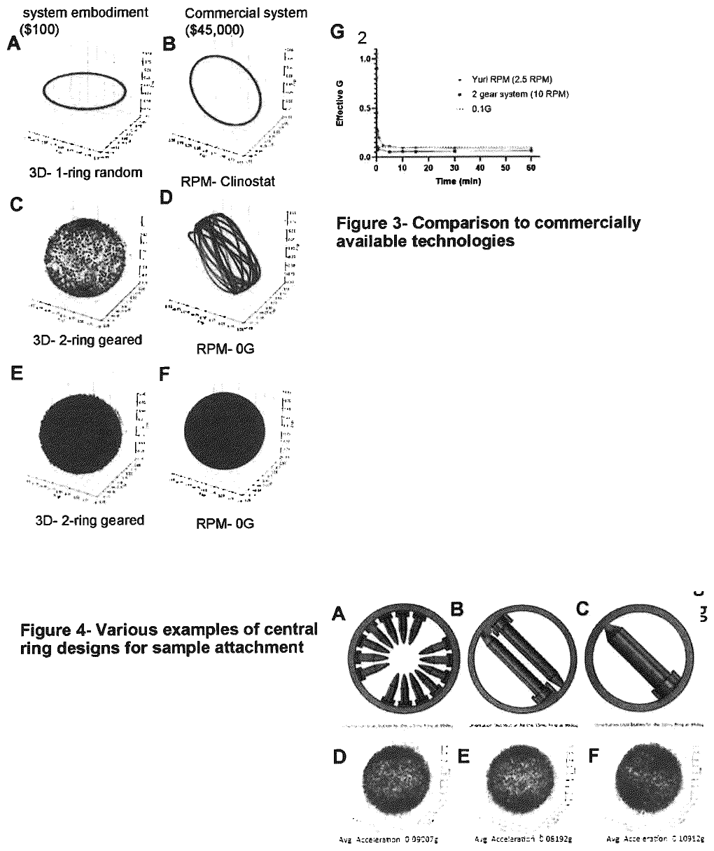

FIGS. 3A-G illustrate a comparison of an embodiment to commercially available technologies, with vector positional data measured using an accelerometer attached at the sample origin on the 1-ring random 3D printed RPM with a dual-falcon tube central ring (FIG. 3, panel A), or on the sample stage of a commercially available RPM set to clinostat mode (10 RPM) (FIG. 3, panel B), or the 2-ring geared 3D printed RPM (10 RPM) (FIG. 3, panel C and FIG. 3, panel E), or the commercially available RPM set to 0 G mode (2.5 RPM) (FIG. 3, panel D and FIG. 3, panel F). FIG. 3, panels A-D show all data points collected in the first 5 minutes, and FIG. 3, panels E, F show data collected after 1 hour. FIG. 3, panel G shows resultant G compared between commercially available RPM set to 0 G mode and a 3D-printed 2-ring system embodiment, measured at various timepoints up to 60 minutes.

In an embodiment, the inner ring is also interchangeable, and enables testing multiple cell environmental conditions and to subsequently test different cell culture flasks with varied media volumes to determine how shear stresses impact cells in a micro-gravity-like environment. FIG. 4 panels A-C show interchangeable inner ring design embodiments for versatile dynamic cell culture systems. In embodiments, the inner ring can be designed to hold any number and sizes of tubes in a variety of configurations. For example, the inner ring can be designed to hold two 15 mL falcon tube as shown in FIG. 4, panel B, one 50 mL falcon tube as shown in FIG. 4, panel C, or up to fourteen 1.5 mL centrifuges as shown in FIG. 4, panel A, according to embodiments.

FIG. 4, panels D-F show vector positional data measured using an accelerometer attached at the indicated red dot on the devices of FIG. 4, panels A-C, respectively. All devices were run at 10 RPM; FIG. 4, panels D-F show all data points collected in the first 15 minutes, and average effective gravity at 15 minutes.

### Biological Proof of Concept Studies: Aggregation and Transfection

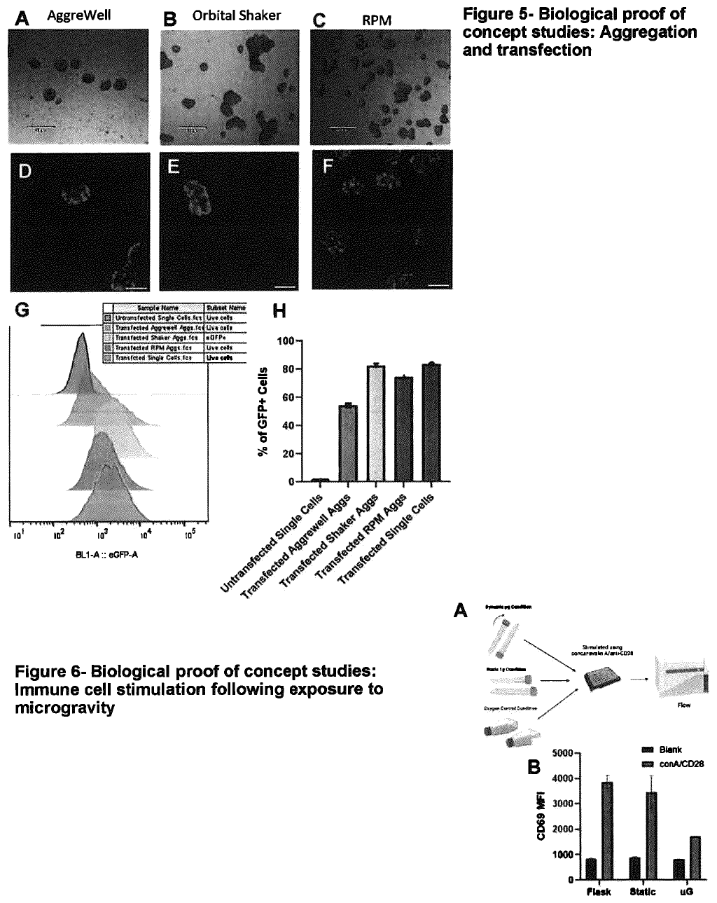

Cellular aggregates are typically challenging to make, and transfection strategies to transfect a whole aggregates or organoids are limited. FIG. 5, panels A-H show successful aggregation and transfection of beta cell aggregates using an embodiment (“RPM”), and a comparison to current technologies (Aggrewell aggregation and Orbital Shaker transfection). FIG. 5, panels A-C show aggregates formed following overnight shaking (scale bar is 320 μm). FIG. 5, panels D-H, shows cells treated with 3500 ng/ml of eGFP mRNA in lipid nanoparticles, and transfection was analysed by confocal microscopy and flow cytometry. In FIG. 5, panels D-F, the scale bar is 100 μm. The 3D printed random positioning machine of an embodiment outperforms the other systems for combined aggregation and transfection.

### Biological Proof of Concept Studies: Immune Cell Stimulation Following Exposure to Microgravity

FIG. 6, panel A is a schematic of cellular activation studies performed in a microgravity device embodiment, under static gravity conditions and using oxygen control conditions. FIG. 6, panel B shows a comparison of CD69 expression in T cells stimulated in microgravity, under static gravity conditions and using oxygen control conditions. In microgravity, T cells show reduced activation, suggesting a more immunosuppressive phenotype. This data is similar to that already published in other microgravity systems and validates the device embodiment is working. It also suggests the device could be used for expansion of regulatory or immunosuppressive cell types.

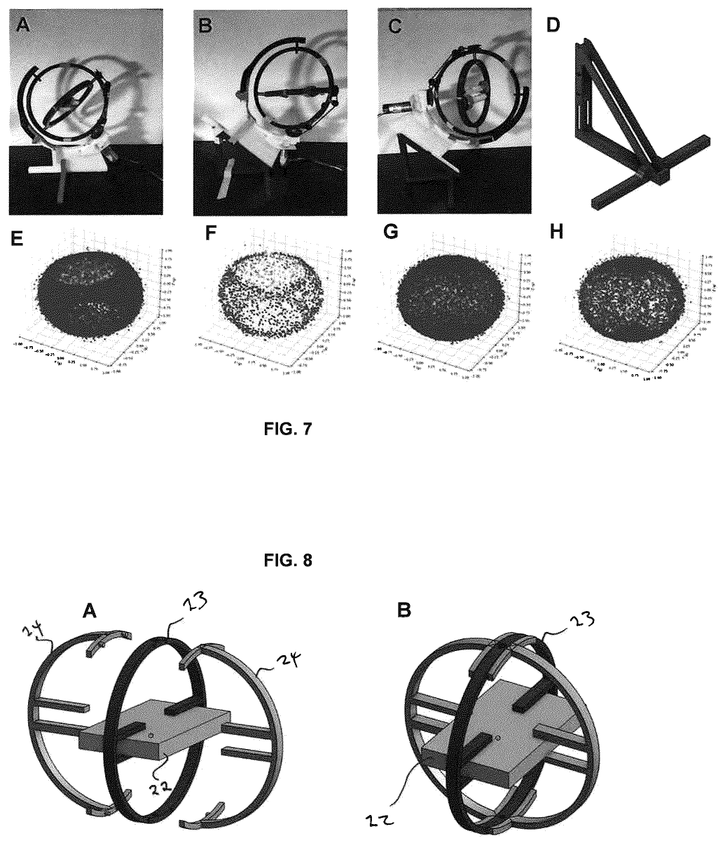

In an embodiment, the effective G experienced may be modified or controlled using different stand configurations according to embodiments. FIG. 7 shows images of different configurations for a microgravity device using different stand configurations according to embodiments. The addition of a stand modifies the effective G experienced by samples within the ring. FIG. 7, panel A shows a normal (45°) stand configuration, panel B shows a modified configuration with motor down (e.g., 0°), and panel C shows a modified configuration with motor up (e.g., 90°). Modified stands facilitate angular changes in the rings within the device which facilitates achieving even lower G. FIG. 7, panel D shows a CAD image of an angled track that allows for the example configurations in FIG. 2, panels B and C. FIG. 7, panels E-H show graphical representations of resultant accelerations with a normal (45°) stand and a modified (90°) stand. Data was collected using a WitMo accelerometer placed between two 15 mL Falcon tubes within a 2-ring system and then organized and analyzed using Excel and Python. The device was run at 10 and 15 RPM with each stand for at least 30 minutes. All graphical representations shown here exclude data from the first 5 minutes of each run as the device gets up to speed and as its motion becomes smoother. The motor used with the current device is a 12V, (max.) 60 RPM motor. FIG. 7, panel E shows a run at 10 RPM with a Normal (45°) stand from t=5 minutes to t=60 minutes; calculated mean g was 0.038 g. FIG. 7, panel F shows a run at 15 RPM with a normal (45°) stand from t=5 minutes to t=30 minutes; calculated mean g was 0.301 g. FIG. 7, panel G shows a run at 10 RPM with a modified (90°) stand from t=5 minutes to t=60 minutes; calculated mean g was 0.027 g. FIG. 7, panel H shows a run at 15 RPM with a modified (90°) stand from t=5 minutes to t=60 minutes; calculated mean g was 0.019 g. 0.019 G is approximately 2% of Earth normal gravity.

FIG. 8 shows a design for an inner ring 20 with a sample holding structure 22, e.g., plate or block-shaped holder, integrated in the inner ring, according to an embodiment. In an embodiment, the sample holding structure may include one or more components. For example, in the embodiment as shown, each of the three components may be printed or otherwise formed separately and then assembled via male-female connectors on each of the side rings and central feature. The holding structure 22 will be placed within the protruding segments around the center of each arc. Four support points should negate any differences due to the weight distribution of a rectangular vessel. The rectangle in the image above represents a vessel/holding structure 22 (e.g., a micro well structure such as a 96-well plate with dimensions of 12.5 cm by 8 cm by 1.5 cm). The ring 23 represents the central ring with the arcs 24 depicting the peripheral components that will be added once the plate has been centered. In the embodiment as shown, the entire structure is designed to be compatible with current 2-ring system, and should be able to be quickly swapped with the inner ring, but may also be used with one ring and three ring embodiments. FIG. 8, panel A shows separated components and panel B shows a combination of all 3 components along male-female connectors.

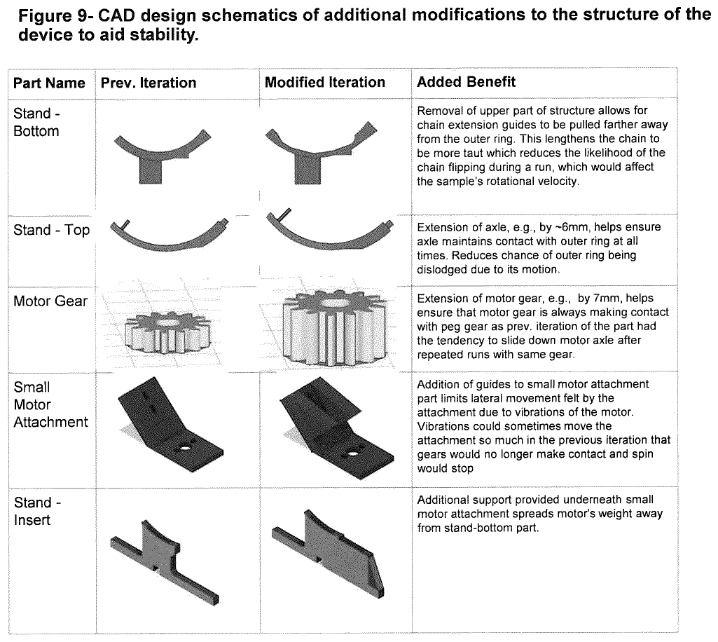

FIG. 9 shows design schematics of various possible example structural modifications to aid stability.

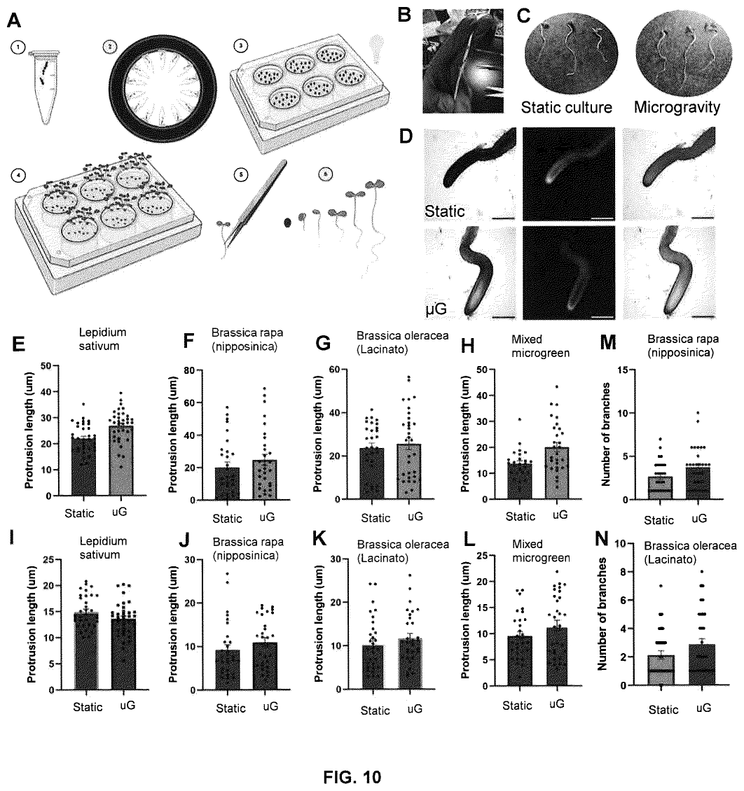

FIG. 10 illustrates aspects of a biological proof of concept study that shows seed treatment in microgravity enhances crop biomass and root growth across species. Seeds were grown on a nutrient-rich substrate, e.g., nutrient soaked fiber mat, and exposed to simulated microgravity (˜0.03 G, 18 hrs 10 RPM) within the device with ambient light conditions. Seeds were then transferred to fiber matting and allowed to grow under normal Earth gravity conditions for a further 48 hours before harvesting. Seedlings were imaged and weighed. Characteristics including root length, shoot length, number of roots and root offshoots and number of leaves were recorded. Panel A shows a schematic of growth protocol. FIG. 10, panel B shows seeds embedded in fiber matting for microgravity stimulation. FIG. 10, panel C shows brightfield images of mixed microgreens taken 48 hours after 18-hour germination phase in Earth-norm gravity or simulated microgravity. FIG. 10, panel D shows brightfield and fluorescent images of roots of Brassica rapa var. nipposinica seedlings grown in normal or simulated microgravity for 18 hours before planting. Images taken 48 hours after return to Earth-norm gravity. Roots were stained with propidium iodide. Scale bar: 500 microns FIG. 10, panels E-N show a variety of edible crops that were studied: seeds from mixed microgreens [mix of Brassica oleracea var. palmifolia, Brassica rapa subsp. chinensis, Brassica rapa var. nipposinica, Brassica oleracea var. capitata f. rubra] and separately, garden cress lepidium sativum, chinese white stem bok choy Brassica rapa chinensis,—mizuna mustard greens/Japense mustard greens Brassica rapa var. nipposinica, and dinosaur kale (Brassica oleracea Lacinato) were treated and analysed as described. Error bars are SEM. Approximately 30-60 seeds were tested in each condition. In brief, seed exposure to microgravity within the device before planting significantly increases root length and root off-shoots in some species. This increase in biomass should be useful for stabilizing plants in varied soil terrain and may increase carbon capture of plants without compromising crop yield.

Use case: Space exploration and commercialized space travel are both increasing in frequency and mission duration. However, cellular behavior is altered during space travel and there are limited tools to study cellular changes in space microenvironments on earth, and then develop technologies to protect astronauts from harm during space travel. For instance, one technology currently available to study the effect of microgravity on cell behavior is a single-axis rotational cell culture system (RCCS). This system does not allow cells to move in multiple axis, is limited in the cell culture flasks that can be studied, and does not allow for simultaneous control or study of other important features such as fluid flow rates, shear stress etc. The present embodiments advantageously provide several novel advancements and new features when compared to the single-axis RCCS system currently commercially available, and may be useful for other applications.

Appendix A of U.S. Provisional Application No. 63/498,105 provides additional aspects and features of embodiments of an RCCS. Appendix A is incorporated by reference herein. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A rotary cell culture system (RCCS), comprising up to three concentric rings, wherein an inner ring is configured to hold one or more cell culture tubes, and wherein each ring rotates around an axis orthogonal to the axes of the other rings.

2. The RCCS of claim 1, wherein the inner ring is removable and interchangeable.

3. The RCCS of claim 1, further comprising a DC motor coupled to an outer ring via one or more gears.

4. The RCCS of claim 3, further comprising a speed controller configured to control operation of the DC motor and rotation of the three rings.

5. The RCCS of claim 1, wherein the up to three concentric rings are attached to each other at opposing axes connection points.

6. The RCCS of claim 1, wherein an outer ring of the up to three concentric rings is attached to a frame along an axis of rotation of the outer ring.

7. The RCCS of claim 6, further including a DC motor coupled to the outer ring via one or more gears and configured to rotate the outer along an axis of the outer ring.

8. A rotary cell culture system (RCCS), comprising at least one concentric ring attached to a frame along an axis of rotation of the at least one concentric ring, and a DC motor coupled to the at least one concentric ring via one or more gears and configured to rotate the at least one concentric ring along the axis.

9. The RCCS of claim 8, wherein the at least one concentric ring is configured to hold one or more cell culture tubes.

10. The RCCS of claim 8, comprising at least two concentric rings, wherein each ring of the at least two concentric rings is attached to an adjacent ring such that the axis of rotation of the at least one concentric ring is orthogonal to an axis of rotation of any other of the concentric rings.

11. The RCCS of claim 10, wherein an inner ring of the at least two concentric rings is configured to hold one or more cell culture tubes.

12. The RCCS of claim 11, wherein the inner ring is removable and interchangeable.

13. The RCCS of claim 11, comprising three concentric rings.

14. A method of processing biological material in a microgravity environment using a rotary cell culture system (RCCS) according to claim 8.

15. A method of processing biological material in a microgravity environment using a rotary cell culture system (RCCS) having at least one ring configured to rotate about a first axis, the method comprising:
placing a biological sample within the at least one ring; and
rotating the at least one ring about the first axis.

16. The method of claim 15, wherein the placing includes placing the biological sample proximal to a center point of the at least one ring, or proximal to a periphery of the at least one ring.

17. The method of claim 15, wherein the RCCS further includes a second ring configured to rotate about a second axis orthogonal to the first axis, wherein a diameter of the second ring is greater than a diameter of the first ring, and wherein the first ring is connected to the second ring at opposing axes connection points, wherein rotating the at least one ring includes rotating the second ring.

18. The method of claim 15, wherein the RCCS further includes a second ring and a third ring configured to rotate about second and third axes, respectively, wherein the second axis is orthogonal to both the first axis and the second axis, wherein a diameter of the third ring is greater than a diameter of the first ring and a diameter of the second ring, and wherein the diameter of the second ring is greater than the diameter of the first ring, and wherein the first ring is connected to the second ring at opposing axes connection points, wherein the second ring is connected to the third ring at opposing axes connection points, wherein the third ring is connected to a frame at opposing axes connection points, and wherein rotating the at least one ring includes rotating the third ring.

19. The method of claim 15, wherein the biological sample includes one of a seed, plant material, or a seed and a nutrient rich substrate.

20. The RCCS of claim 1, further including a sample holding structure located proximal to a center of the inner ring and held by a structure coupled with or integrated into the inner ring.

21. The RCCS of claim 10, wherein an inner ring of the at least two concentric rings is configured to hold a sample holding structure proximal to a center of the inner ring.

22. The RCCS of claim 21, wherein the sample holding structure includes a micro well plate structure.

23. The RCCS of claim 8, further including an adjustable stand with connector elements configured to change an angle of the at least one concentric ring.
